(19) United States
(12) Patent Application Publication
(10) Pub. No.: US 2026/0167932 A1
(43) Pub. Date: Jun. 18, 2026

(54) METHOD TO GENERATE IMMUNO-FREE AND STRESS-RESISTANT DONOR CELLS

(71) Applicant: Reem MAHRAT, Pleasanton, CA (US)

(72) Inventor: Reem MAHRAT, Pleasanton, CA (US)

(21) Appl. No.: 18/985,962

(22) Filed: Dec. 18, 2024

(51) Int. Cl. C12N 5/0783; A61K 35/12; A61K 48/00; A61P 37/02; C12N 5/0781; C12N 9/22; C12N 15/113

(52) U.S. Cl. CPC C12N 5/0637; A61K 35/12; A61K 48/0041; A61K 48/0083; A61P 37/02; C12N 5/0635; C12N 9/226; C12N 15/113; C12N 2310/20; C12N 2510/02

(57) ABSTRACT

A method for generating immuno-free and stress-resistant donor cells comprising editing PRDM1 and FOXP3 genes in T-cells and B-cells with CRISPR-Cas9 and modulating immune responses to reduce inflammation and prevent autoimmunity. Particularly, the present invention discloses a method for treating Type 1 Diabetes, comprising genetically modifying pancreatic beta cells with CRISPR-Cas9, and altering HLA expression, and upregulating immune checkpoint molecules such as PD-L1 to obtain immune-tolerant and stress-resistant pancreatic beta cells.

### FIELD OF THE INVENTION

The present disclosure relates to a method to generate immuno-free and stress-resistant donor cells. More specifically, the present disclosure relates to a comprehensive therapeutic method for autoimmune diseases utilizing gene editing, epigenetic modulation, and immune-tolerant stem cells to generate immuno-free and stress-resistant donor cells via CRISPR-Cas9.

### BACKGROUND

Autoimmune diseases, such as lupus, multiple sclerosis, type 1 diabetes, and rheumatoid arthritis, involve the immune system mistakenly attacking the body's own tissues. Existing therapies mainly focus on symptomatic relief rather than addressing the root causes. Advances in gene-editing technologies, epigenetic modulation, and stem cell research offer the potential to not only modulate immune responses but also regenerate damaged tissues.

Gene editing is a set of strategies used to modify the expression of an individual's genes or to generate immuno-free and stress-resistant donor cells. Cell therapy is the administration of live cells or maturation of a specific cell population in a patient for the treatment of a disease. Gene editing and cell therapy are overlapping fields, with the goals of targeting the cause of diseases in the nucleic acid or cellular population. For example, hematopoietic diseases can be treated by transplantation of ex vivo gene-modified stem cells (e.g., hematopoietic stem/progenitor cells and hematopoietic stem cells, also referred to herein as HSCs) into a subject.

The discovery and application of the CRISPR/Cas9 system in mammalian cells results in effective and precise editing of target genes, e.g., through the non-homologous end joining pathway (NHEJ), homology-directed repair (HDR), or other DNA repair pathways. Co-delivery of a Cas9 molecule and a target-specific guide RNA (gRNA) molecule, optionally along with a donor DNA repair template molecule, facilitates gene-editing of a target sequence (e.g., a disease-related mutation) in the genome. Thus, the use of the CRISPR/Cas9 system for genes is a promising strategy for treating multiple genetic disorders. However, stem cells are extremely sensitive to manipulation in vitro and ex vivo, and, thus, manipulation of stem cells using CRISPR/Cas9 systems, to date, has been inefficient and has resulted in little, if any, long-term viability and engraftment of the stem cells in vivo.

Many patients in need of organ/tissue transplantation are already battling chronic diseases. In autoimmune diseases, the immune cells/antibodies of patients turn against their own cells, posing a significant challenge in organ/tissue transplantation as the universal donor cells are also targeted post-transplantation. Therefore, we are on the brink of a revolutionary advancement in transplantation science. We are further working to transform the universal donor cells (immuno-free donor cells) into stress-resistant cells that can withstand harsh in vivo environments, thereby significantly enhancing the success of donor tissue/organ survival.

Therefore, there is a need to address the above-mentioned challenges by generating an immuno-free donor cell for non-autoimmune diseases and generating immuno-free and stress-resistant donor cells for autoimmune diseases.

Hence, the present disclosure provides a comprehensive, multi-pronged approach to treating autoimmune diseases.

### SUMMARY

The present invention provides a method to generate immuno-free and stress-resistant donor cells, which could be used for universal tissue/organ transplantation. The method comprises a multi-faceted therapeutic approach that targets and corrects immune dysregulation through precise gene editing of immune cells.

In an embodiment, the present invention enhances immune tolerance post-transplantation by promoting the function of regulatory T cells.

In an embodiment, the present invention introduces immune-tolerant stem cells to regenerate damaged tissues while avoiding immune rejection post-transplantation.

In an embodiment, the present invention leverages exosome-based delivery systems for precision targeting, minimizing side effects, and improving cell therapy efficacy.

In an embodiment, the present invention provides comprehensive approach addresses both the causes and effects of autoimmune diseases, offering potential remission and tissue repair in affected patients. In an embodiment, the present invention provides a method for generating immuno-free and stress-resistant donor cells comprising editing PRDM1 and FOXP3 genes in T-cells and B-cells with CRISPR-Cas9 and modulating immune responses to reduce inflammation and prevent autoimmunity.

In an aspect of the present invention, the PRDM1 gene is activated to produce BLIMP-1 proteins for immune cell differentiation and function for T and B cells.

In an aspect of the present invention, the CRISPR-Cas9 edits the PRDM1 gene in immune cells, particularly T cells and B cells, for decreasing the inflammatory response and curbing the progression of autoimmune diseases.

In an aspect of the present invention, autoimmune diseases are selected from multiple sclerosis, type 1 diabetes, lupus and rheumatoid arthritis.

In an aspect of the present invention, the modulation is an epigentic modulation to fine-tune PRDM1 expression by RNA interference (RNAi) or other epigenetic to allow dynamic regulation of immune cell activity as needed without causing permanently silencing the gene to save its physiological function as much as possible

In an aspect of the present invention, the CRISPR Activation (CRISPRa) upregulates FOXP3 expression in Tregs, enhancing their ability to suppress harmful immune responses, thereby reducing autoimmunity in diseases.

In an aspect of the present invention, the CRISPR-Cas9 technologymutates HLA (human leukocyte antigen) genes of stem cells at a proteomic level to reduce the risk of immune rejection post-transplanation.

In an aspect of the present invention, the stem cells are modified to express immune-regulatory molecules such as CTLA-4 and PD-L1 to create an immune-tolerant phenotype.

In an aspect of the present invention, the immune-tolerant stem cells are engineered to differentiate into specific cell types needed to regenerate tissues damaged by autoimmune attacks, selected from insulin-producing beta cells for type 1 diabetes or myelin-producing cells for multiple sclerosis.

In an aspect of the present invention, the stem cells secrete immunomodulatory cytokines selected from IL-10, TGF-β to promote immune tolerance and reduce local inflammation, enhancing the overall therapeutic effect.

In an aspect of the present invention, the process comprises delivering exosomes derived from mesenchymal stem cells, CRISPR-Cas9 gene editing tools, and immune-tolerant stem cells directly to target tissues, ensuring precise and effective delivery while minimizing systemic side effects.

In an aspect of the present invention, the present invention provides that the exosomes are engineered to carry specific targeting molecules selected from antibodies or ligands to recognize and bind to inflamed tissues or affected organs, allowing the gene-editing and stem cell therapies to reach their intended sites of action with high specificity.

In an aspect of the present invention, the combination of PRDM1 and FOXP3 gene editing reprograms the patient's immune system, reducing the likelihood of autoimmune attacks by promoting immune tolerance while still allowing for normal immune responses to infections or other threats.

In an aspect of the present invention, the present invention provides a system for generating immuno-free and stress-resistant donor cells comprising HLA genes incorporated with immune-regulatory molecules selected from CTLA-4 and PD-L1, allowing for safe tissue regeneration and long-term immune evasion in autoimmune disease patients.

In an aspect of the present invention, the present invention provides a method of delivering gene-editing components and immune-tolerant stem cells via exosome-mediated targeting, ensuring precise delivery to inflamed or damaged tissues and minimizing systemic exposure and side effects.

In an aspect of the present invention, the present invention provides a combination therapy for autoimmune diseases, utilizing gene editing to reprogram immune cells and immune-tolerant stem cells for tissue regeneration, resulting in both immune modulation and tissue repair.

In an aspect of the present invention, the present invention provides a method for treating Type 1 Diabetes, comprising genetically modifying pancreatic beta cells with CRISPR-Cas9 and altering HLA expression and upregulating immune checkpoint molecules such as PD-L1 to obtain immune tolerant pancreatic beta cells.

In an aspect of the present invention, the beta cells are genetically engineered with CRISPR-Cas9 to modify key immune recognition genes, specifically HLA class I and II molecules, reducing their visibility to the immune system.

In an aspect of the present invention, the edited beta cells express PD-L1, an immune checkpoint molecule that suppresses immune attacks by engaging with the PD-1 receptor on T cells.

In an aspect of the present invention, controlling the expression of other immune-modulatory genes allows for dynamic regulation of immune response, particularly in response to changing immune conditions by RNAi or Epigenetic Modulation tools.

In an aspect of the present invention, the method further comprises the fact that the beta cells are engineered to maintain their insulin production capabilities, ensuring that they can effectively respond to glucose levels in the body.

In an aspect of the present invention, the beta cells are fine-tuned with CRISPR technology to enhance their sensitivity to glucose fluctuations, optimizing insulin secretion and improving glycemic control in patients with Type 1 Diabetes.

In an aspect of the present invention, delivering the immune-tolerant beta cells to the pancreas using exosome-based delivery systems.

In an aspect of the present invention, the exosomes are engineered with surface ligands that recognize inflamed pancreatic tissues, ensuring that the immune-tolerant beta cells are delivered to areas most affected by autoimmune destruction.

In an aspect of the present invention, a system for delivering immune-tolerant beta cells using exosome-based targeting mechanisms, ensuring precise delivery to the pancreatic tissue and minimizing systemic immune response.

In an aspect of the present invention, the present invention provides a method for regulating immune response in Type 1 Diabetes patients, utilizing immune-tolerant beta cells that secrete immunomodulatory cytokines to create a local immune-tolerant environment and prevent further destruction of insulin-producing cells.

In an aspect of the present invention, the present invention provides a method of optimizing insulin production in genetically modified beta cells, using CRISPR to enhance glucose responsiveness, ensuring efficient insulin secretion in response to blood sugar levels.

### BRIEF DESCRIPTION OF DRAWINGS

FIG. 1 illustrates how immuno-free donor cells increase donor tissue/organ survival after transplantation.

FIG. 2 illustrates how immuno-free and stress-resistant donor cells further increase donor tissue/organ survival by resisting harsh in vivo environment.

FIG. 3a illustrates pathological in vivo environment: Autoimmune diseases and

FIG. 3b illustrates immuno-free & stress-resistant donor cells.

FIG. 4a illustrates pathological in vivo environment: Chronic disease and

FIG. 4b illustrates immuno-free & stress-resistant donor cells.

### DETAILED DESCRIPTION

In order to describe features of the disclosure, a more particular description of the presently described technology is rendered by reference to specific embodiments thereof, which are illustrated in the appended drawings. Understanding that these drawings depict only example embodiments of the disclosure and are not, therefore, to be considered to be limiting in their scope, the principles herein are described and explained with additional specificity and detail through the use of the accompanying drawings.

The present disclosure provides a method to generate immuno-free and stress-resistant donor cells using multiple cutting-edge technologies. It combines CRISPR-Cas9 gene editing, epigenetic regulation, and immune-tolerant stem cell therapy to provide a powerful, long-lasting solution to autoimmune diseases.

The present disclosure provides a comprehensive, multi-pronged approach to treating autoimmune diseases by combining cutting-edge CRISPR-Cas9 gene editing, epigenetic modulation, and immune-tolerant stem cell technology. By tackling the root causes of autoimmunity, promoting immune tolerance, and enabling tissue regeneration, the present disclosure represents a significant advancement in the field of precision medicine for autoimmune diseases.

The method of the present invention is explained in the below points:

### 1. PRDM1 and FOXP3 Gene Editing for Autoimmune Modulation

PRDM1 Targeting: The PRDM1 gene, when activated, produces BLIMP-1 proteins, which are essential for immune cell differentiation and function, particularly for T and B cells. Aberrations in PRDM1 expression led to dysregulated immune responses.
CRISPR-Cas9 is used to precisely edit the PRDM1 gene in immune cells (T cells and B cells), decreasing the inflammatory response and curbing the progression of autoimmune diseases such as multiple sclerosis, type 1 diabetes, lupus, and rheumatoid arthritis.
Epigenetic Modulation: To fine-tune PRDM1 expression, RNA interference (RNAi) or other epigenetic tools is employed, allowing dynamic regulation of immune cell activity without permanently silencing the gene.
FOXP3 Enhancement for Regulatory T-Cell Function: FOXP3 is critical in the development and function of regulatory T cells (Tregs), which help in maintaining immune tolerance by preventing auto-reactive immune responses.
CRISPR Activation (CRISPRa) is used to upregulate FOXP3 expression in Tregs, enhancing their ability to suppress harmful immune responses, thereby reducing autoimmunity in diseases like type 1 diabetes and multiple sclerosis.
This approach ensures that auto-reactive immune cells are suppressed while allowing normal immune function to continue.

### 2. Development of Immune-Tolerant Stem Cells

Creation of Immune-Evasive Properties: Using CRISPR-Cas9 technology, the HLA (human leukocyte antigen) genes of stem cells is edited to reduce the risk of immune rejection. These stem cells are further modified to express immune-regulatory molecules (e.g., CTLA-4, PD-L1) to create an immune-tolerant phenotype.
Stem Cells for Tissue Regeneration: The immune-tolerant stem cells are engineered to differentiate into specific cell types needed to regenerate tissues damaged by autoimmune attacks, such as insulin-producing beta cells for type 1 diabetes or myelin-producing cells for multiple sclerosis.
Immune Modulation: These stem cells are designed to secrete immunomodulatory cytokines (e.g., IL-10, TGF-β) that promote immune tolerance and reduce local inflammation, enhancing the overall therapeutic effect.
Dual Function: These immune-tolerant stem cells not only replace damaged tissues but also act as long-term modulators of the immune system, continuously working to suppress autoimmune responses normally.

### 3. Exosome-mediated Delivery System

Exosome-Based Delivery: Using exosomes derived from mesenchymal stem cells, CRISPR-Cas9 gene editing tools and immune-tolerant stem cells are delivered directly to target tissues, ensuring precise and effective delivery while minimizing systemic side effects.
Targeted Therapy: The exosomes are engineered to carry specific targeting molecules (e.g., antibodies or ligands) that recognize and bind to inflamed tissues or affected organs, allowing the gene-editing and stem cell therapies to reach their intended sites of action with high specificity.

### 4. Combinatorial Therapy for Long-Term Remission

Immune System Reprogramming: The combination of PRDM1 and FOXP3 gene editing reprograms the patient's immune system, reducing the likelihood of autoimmune attacks by promoting immune tolerance while still allowing for normal immune responses to infections or other threats.
Tissue Regeneration and Repair: The introduction of immune-tolerant stem cells allows for the repair of tissues that have been damaged by autoimmune processes. These cells, due to their immune-evasive properties, are not attacked by the patient's immune system, even in cases where the patient has developed autoimmunity to those tissues.
Sustained Autoimmune Suppression: By enhancing the regulatory function of Tregs and suppressing pro-inflammatory immune cells, this therapy provides long-term suppression of autoimmunity, potentially leading to sustained remission or even a cure in certain autoimmune diseases.

The potential impact of the present invention is to provide long-term remission by addressing both immune dysregulation and tissue damage; this treatment has the potential to provide long-term remission or even a cure for many autoimmune diseases.

Further, the present invention provides a way to achieve personalized Medicine by which the therapy can be tailored to individual patients, with gene editing and stem cell modifications customized based on their specific genetic and immune profiles, supporting the principles of precision medicine.

Moreover, the present invention provides tissue regeneration and repair in addition to modulating the immune system. This therapy enables the regeneration of tissues damaged by autoimmune attacks, providing dual benefits: symptom alleviation and functional recovery.

Further, the restoration of natural insulin production in patients with Type 1 diabetes would no longer need external insulin injections, as the immune-tolerant beta cells would functionally replace the lost beta cells, restoring normal glucose regulation.
Immune Protection: The immune-tolerant properties of the beta cells would prevent further immune-mediated destruction, providing long-term protection against autoimmune attacks.

Further the present method has the potential to offer long-term remission for T1D patients, allowing them to lead normal lives without constant glucose monitoring and insulin therapy.

Hence, the present disclosure provides incorporating immune-tolerant beta cells and offers a comprehensive, multi-pronged approach to treating Type 1 Diabetes. By addressing both the root cause (autoimmune destruction) and the symptom (loss of insulin production), this therapy provides a revolutionary solution that has the potential to transform the standard of care for patients suffering from this debilitating autoimmune disease.

FIG. 1 illustrates how immuno-free donor cells increase donor tissue/organ survival after transplantation. The immuno-free donor cells from the donor are transplanted to recipient's normal in vivo environment. In the normal in vivo environment, recipient's immuno-cells and antibodies attack foreign cells and destroy non-immuno-free donor cells.

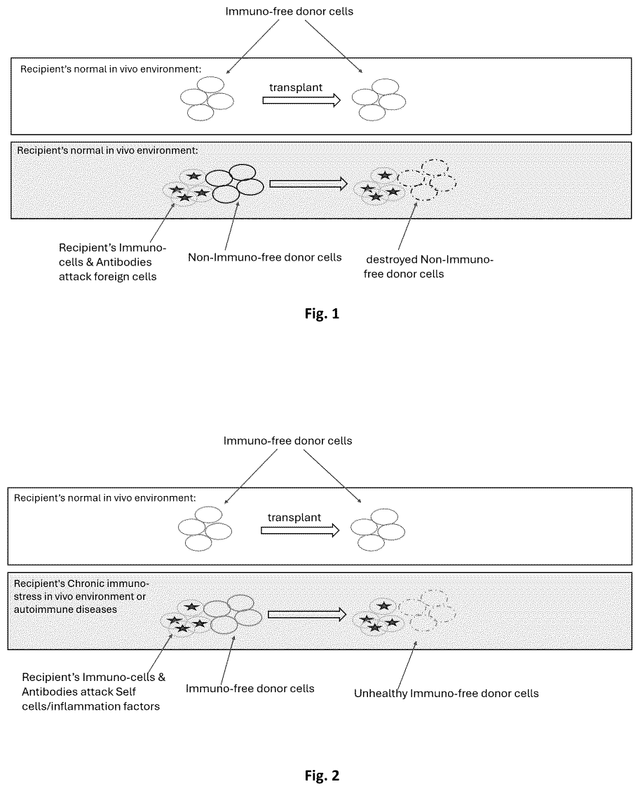

FIG. 2 illustrates generating immuno-free and stress-resistant donor cells to further increase donor tissue/organ survival by resisting harsh in vivo environments. The immuno-free donor cells from the donor are transplanted to recipient's normal in vivo environment. In the recipient's chronic immuno-stress in vivo environment or autoimmune diseases, the recipient's immuno-cells and antibodies attack self-cells/inflammation factors, making them unhealthy immuno-free donor cells.

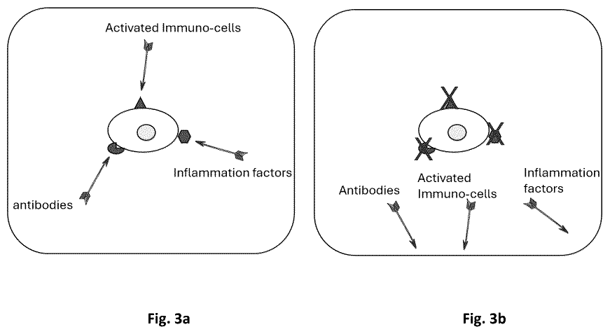

FIG. 3a illustrates Pathological in vivo environment: Autoimmune diseases. In the figure, it is shown that antibodies and inflammation factors attack the Immuno-free donor cells with activated immune cells. FIG. 3b illustrates that Immuno-free & stress-resistant donor cells would not be attacked by the antibodies, activated immune cells, and inflammation factors.

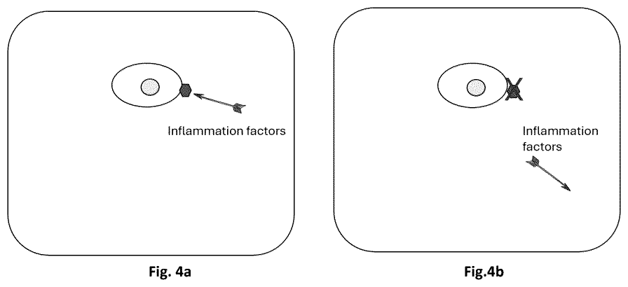

FIG. 4a illustrates the pathological in vivo environment of chronic disease. FIG. 4a illustrates that Immuno-free donor cells would be attacked by inflammation factors, and

FIG. 4b illustrates that Immuno-free & stress-resistant donor cells would not be attacked by inflammation factors.

### Example 1

Type 1 Diabetes (T1D): Immune-tolerant beta cells are specialized types of pancreatic beta cells that has been genetically modified to evade immune system attacks. The cells are engineered to both retain their normal function of producing insulin in response to blood glucose levels and to avoid being destroyed by the body's immune system, which is the hallmark of autoimmune diseases like Type 1 Diabetes (T1D). In T1D, the immune system mistakenly identifies beta cells as harmful and attacks them, leading to insulin deficiency.

To make immune-tolerant beta cells, genetic modifications are made to reduce their

visibility to the immune system, prevent immune activation, and create a local immune-suppressive environment.

Here are the characteristics of immune-tolerant beta cells:

### 1. Insulin Production and Glucose Responsiveness

Beta cells are naturally responsible for producing insulin, the hormone that regulates blood glucose levels.
In their immune-tolerant form, these cells are engineered to maintain or enhance their ability to detect blood glucose levels and secrete insulin accordingly.

### 2. Immune Evasion Capabilities

HLA Modification: HLA (human leukocyte antigen) genes are involved in presenting proteins to immune cells, essentially allowing the immune system to “see” the cells. By editing the HLA class I and II genes, immune-tolerant beta cells reduce the likelihood of being recognized and targeted by auto-reactive T-cells, which are the immune cells that attack beta cells in T1D.
Expression of Immune Checkpoint Molecules: Immune-tolerant beta cells are engineered to express immune checkpoint molecules like PD-L1. PD-L1 interacts with the PD-1 receptor on T-cells to suppress their activity. This checkpoint mechanism effectively tells the immune cells to “stand down,” preventing them from attacking the beta cells.

### 3. Anti-inflammatory and Immune-Modulatory Properties

Cytokine Secretion: Immune-tolerant beta cells can be further engineered to secrete anti-inflammatory cytokines, such as IL-10 or TGF-β, which help suppress local immune responses. This creates an immune-privileged environment around the beta cells, making it harder for the immune system to mount an attack.
Epigenetic Modulation: Additional layers of immune tolerance can be achieved through epigenetic tools (like RNA interference), allowing the immune-tolerant beta cells to dynamically adjust their immune-modulatory behavior based on the patient's immune activity.

### 4. Engineered Resilience

CRISPR-Cas9 Modifications: Gene-editing techniques such as CRISPR-Cas9 are used to precisely alter the beta cells, introducing both immune-evasive properties and ensuring that the beta cells retain their essential function of insulin production.

In summary, immune-tolerant beta cells are engineered to overcome the immune challenges faced by beta cells in autoimmune diseases, particularly Type 1 Diabetes. They are designed to function like normal beta cells in terms of insulin production but are armed with genetic modifications that allow them to evade immune system attacks, providing long-lasting and potentially curative therapy for autoimmune conditions like T1D.

Immune-tolerant beta cells are specialized insulin-producing cells engineered to both evade immune system attacks and restore normal insulin production in patients with Type 1 Diabetes (T1D). The immune system in T1D patients erroneously targets and destroys pancreatic beta cells, leading to insulin deficiency and glucose dysregulation. Current treatments focus on insulin replacement but do not address the underlying autoimmune attack or the body's inability to produce insulin naturally.

This novel approach introduces immune-tolerant beta cells, leveraging CRISPR-Cas9 technology and immune-modulatory features to provide a long-term solution to T1D by both preventing immune destruction and restoring insulin production.

### 1. Development of Immune-tolerant Beta Cells

CRISPR-Cas9 Gene Editing for Immune Evasion: The beta cells are genetically engineered using CRISPR-Cas9 to modify key immune recognition genes, specifically HLA class I and II molecules, reducing their visibility to the immune system. This helps beta cells evade detection and destruction by auto-reactive T-cells that mistakenly target insulin-producing cells in T1D patients.
PD-L1 Expression: The edited beta cells express PD-L1, an immune checkpoint molecule that suppresses immune attacks by engaging with the PD-1 receptor on T cells. This prevents immune cells from recognizing and attacking the beta cells, creating an immune-tolerant environment.

### 2. Epigenetic Modulation for Immune Tolerance

RNAi or Epigenetic Modulation tools are used to control the expression of other immune-modulatory genes, allowing for dynamic regulation of immune response, particularly in response to changing immune conditions in the patient's body. This ensures that immune tolerance is maintained over time without permanent suppression of immune function.

### 3. Beta Cell Functionality and Insulin Production

In addition to immune-tolerant properties, the beta cells is engineered to maintain their insulin production capabilities, ensuring that they can effectively respond to glucose levels in the body.
Gene Editing for Enhanced Glucose Responsiveness: Using CRISPR technology, beta cells can be fine-tuned to enhance their sensitivity to glucose fluctuations, optimizing insulin secretion and improving glycemic control in patients with Type 1 Diabetes.

### 4. Exosome-Mediated Delivery of Beta Cells

The immune-tolerant beta cells are delivered to the pancreas using exosome-based delivery systems. These exosomes, derived from mesenchymal stem cells, carry the beta cells directly to the pancreatic environment, ensuring precise targeting and reducing off-target effects.
Exosome Targeting Mechanism: The exosomes are engineered with surface ligands that recognize inflamed pancreatic tissues, ensuring that the immune-tolerant beta cells are delivered to areas most affected by autoimmune destruction.

### 5. Dual Function: Immune Tolerance and Tissue Regeneration

Beyond their insulin-producing function, these immune-tolerant beta cells play a role in immune modulation by secreting anti-inflammatory cytokines (e.g., IL-10, TGF-β). This further promotes a local environment of immune tolerance, helping to suppress ongoing autoimmune attacks in the pancreas.
This dual function provides both functional recovery (restoration of insulin production) and long-term disease control (reduced autoimmunity), offering a comprehensive treatment solution for T1D.

### Example 2

The inclusion of immune-tolerant beta cells offers a revolutionary approach for treating Type 1 Diabetes and could potentially be adapted for other autoimmune diseases where cell replacement therapies are critical. Specifically:
Type 1 Diabetes: These engineered beta cells restore natural insulin production while evading immune destruction, offering a potentially curative solution that goes beyond insulin injections.
Autoimmune Pancreatitis: The immune-tolerant beta cells could be adapted to treat autoimmune-related pancreatic conditions, restoring both function and immune balance in the pancreas.
Stem Cell-Based Regenerative Medicine: The same principles can be applied to other organs and tissues, where immune-tolerant cell types could regenerate damaged tissues while avoiding immune rejection.

In view of the above, the method of the present invention is applicable to a broad range of autoimmune diseases such as type 1 Diabetes by introducing immune-tolerant beta cells to restore insulin production, while CRISPR-mediated enhancement of Tregs reduces the autoimmune attack on these cells. In multiple sclerosis, myelin-producing cells are reintroduced to repair damaged nerve sheaths, and auto-reactive immune cells are suppressed through PRDM1 editing and FOXP3 enhancement. Further, the method is utilized in rheumatoid Arthritis, where immune-tolerant stem cells help regenerate joint tissues, while CRISPR editing of immune cells reduces the chronic inflammation that drives disease progression.

1. A method for generating immuno-free and stress-resistant donor cells comprising:
i. editing PRDM1 and FOXP3 genes in T-cells and B-cells with CRISPR-Cas9 and
ii. modulating immune responses to reduce inflammation and prevent autoimmunity.

2. The method, as claimed in claim 1, wherein the PRDM1 gene is activated to produce BLIMP-1 proteins for immune cell differentiation and function, for T and B cells.

3. The method, as claimed in claim 1, wherein the CRISPR-Cas9 edits the PRDM1 gene in immune cells, particularly, T cells and B cells for decreasing the inflammatory response and curbing the progression of autoimmune diseases.

4. The method, as claimed in claim 1, wherein the autoimmune diseases are selected from multiple sclerosis, type 1 diabetes, lupus, and rheumatoid arthritis.

5. The method, as claimed in claim 1, wherein the modulation is an epigentic modulation to fine-tune PRDM1 expression by RNA interference (RNAi) or other epigenetic to allow dynamic regulation of immune cell activity without permanently silencing the gene.

6. The method, as claimed in claim 1, wherein the CRISPR Activation (CRISPRa) upregulates FOXP3 expression in Tregs, enhancing their ability to suppress harmful immune responses and thereby reducing autoimmunity in diseases.

7. The method, as claimed in claim 1, wherein the CRISPR-Cas9 technology edits HLA (human leukocyte antigen) genes of stem cells to reduce the risk of immune rejection.

8. The method, as claimed in claim 7, wherein the stem cells are modified to express immune-regulatory molecules such as CTLA-4and PD-L1 to create an immune-tolerant phenotype.

9. The method, as claimed in claim 1, wherein the immune-tolerant stem cells are engineered to differentiate into specific cell types needed to regenerate tissues damaged by autoimmune attacks, selected from insulin-producing beta cells for type 1 diabetes or myelin-producing cells for multiple sclerosis.

10. The method, as claimed in claim 1, wherein the stem cells secrete immunomodulatory cytokines selected from IL-10and TGF-β to promote immune tolerance and reduce local inflammation, enhancing the overall therapeutic effect.

11. The method, as claimed in claim 1, wherein the process comprises delivering exosomes derived from mesenchymal stem cells, CRISPR-Cas9 gene editing tools, and immune-tolerant stem cells directly to target tissues, ensuring precise and effective delivery while minimizing systemic side effects.

12. The method, as claimed in claim 1, wherein the exosomes are engineered to carry specific targeting molecules selected from antibodies or ligands to recognize and bind to inflamed tissues or affected organs, allowing the gene-editing and stem cell therapies to reach their intended sites of action with high specificity.

13. The method as claimed in claim 1, wherein the combination of PRDM1 and FOXP3 gene editing reprograms the patient's immune system, reducing the likelihood of autoimmune attacks by promoting immune tolerance while still allowing for normal immune responses to infections or other threats.

14. A system for generating immuno-free and stress-resistant donor cells comprising:
HLA genes are incorporated with immune-regulatory molecules selected from CTLA-4 and PD-L1, allowing for safe tissue regeneration and long-term immune evasion in autoimmune disease patients.

15. A method of delivering gene-editing components and immune-tolerant stem cells via exosome-mediated targeting, ensuring precise delivery to inflamed or damaged tissues, minimizing systemic exposure and side effects.

16. A combination therapy for autoimmune diseases, utilizing gene editing to reprogram immune cells and immune-tolerant stem cells for tissue regeneration, resulting in both immune modulation and tissue repair.

17. A method for treating Type 1 Diabetes, comprising:
i. genetically modifying pancreatic beta cells with CRISPR-Cas9 and
ii. Altering HLA expression and upregulating immune checkpoint molecules such as PD-L1 to obtain immune-tolerant pancreatic beta cells.

18. The method, as claimed in claim 17, wherein the beta cells are genetically engineered with CRISPR-Cas9 to modify key immune recognition genes, specifically HLA class I and II molecules, reduces their visibility to the immune system.

19. The method, as claimed in claim 17, wherein the edited beta cells express PD-L1, an immune checkpoint molecule that suppresses immune attacks by engaging with the PD-1 receptor on T cells.

20. The method, as claimed in claim 17, controls the expression of other immune-modulatory genes, allowing for dynamic regulation of immune response, particularly in response to changing immune conditions by RNAi or Epigenetic Modulation tools.

21. The method, as claimed in claim 17, further comprises that the beta cells are engineered to maintain their insulin production capabilities, ensuring that they can effectively respond to glucose levels in the body.

22. The method, as claimed in claim 17, is wherein the beta cells are fine-tuned with CRISPR technology to enhance their sensitivity to glucose fluctuations, optimize insulin secretion and improve glycemic control in patients with Type 1 Diabetes.

23. The method, as claimed in claim 17, involves delivering immune-tolerant beta cells to the pancreas using exosome-based delivery systems.

24. The method, as claimed in claim 17, wherein the exosomes are engineered with surface ligands that recognize inflamed pancreatic tissues, ensuring that the immune-tolerant beta cells are delivered to areas most affected by autoimmune destruction.

25. A system for delivering immune-tolerant beta cells using exosome-based targeting mechanisms, ensuring precise delivery to the pancreatic tissue and minimizing systemic immune response.

26. A method for regulating immune response in Type 1 Diabetes patients, utilizing immune-tolerant beta cells that secrete immunomodulatory cytokines to create a local immune-tolerant environment and prevent further destruction of insulin-producing cells.

27. A method of optimizing insulin production in genetically modified beta cells, using CRISPR to enhance glucose responsiveness, ensuring efficient insulin secretion in response to blood sugar levels.
